(19) United States
(12) Patent Application Publication
(10) Pub. No.: US 2019/0053724 A1
(43) Pub. Date: Feb. 21, 2019

(54) SPHYGMOMANOMETER CUFF FOR FINGER

(71) Applicant: SOCIONEXT INC., Yokohama-shi (JP)

(72) Inventors: Hiroyuki TOMURA, Yokohama-shi (JP); Masato YOSHIOKA, Yokohama-shi (JP); Ryusuke KURACHI, Yokohama-shi (JP); Masaya TAMAMURA, Yokohama-shi (JP); Amane INOUE, Yokohama-shi (JP); Minoru NAKAGAWARA, Sapporo-shi (JP)

(21) Appl. No.: 16/165,562

(22) Filed: Oct. 19, 2018

Related U.S. Application Data
(63) Continuation of application No. PCT/JP2016/062350, filed on Apr. 19, 2016.

(51) Int. Cl. A61B 5/022

(52) U.S. Cl. CPC A61B 5/02241

(57) ABSTRACT

A sphygmomanometer cuff for finger, includes, a base unit including a first curved portion extending along a part of periphery of a finger defined as a measurement examinee's target region, at least one movable unit connected via a rotatable unit to the base unit, including a second curved portion extending along another part of periphery of the finger, and being attachable to the finger in a state of an opening being formed along the periphery of the finger, an adjustment unit configured to adjust an angle of the movable unit to the base unit, and a compression unit provided along at least a part of an inner peripheral portion formed along the periphery of the finger by the base unit, the movable unit and the opening, and compressing the part of periphery of the finger.

### CROSS-REFERENCE TO RELATED APPLICATION

This application is a continuation application of International Application PCT/JP2016/062350, filed on Apr. 19, 2016 and designated the U.S., the contents of which are incorporated herein by reference.

### FIELD

The present application pertains to a sphygmomanometer cuff for finger.

### BACKGROUND

A sphygmomanometer cuff is connected to a sphygmomanometer body and is wound around a measurement examinee's region when measuring a blood pressure. An airbag is provided inside of the sphygmomanometer cuff. The airbag is expanded by supplying air into the airbag, thus measuring the blood pressure of the measurement examinee's region. Among methods of non-invasively measuring the blood pressures, there is a volume compensation method as a method capable of performing consecutive measurements per heartbeat. Attainment of this volume compensation method entails compressing blood vessels of an artery of the measurement examinee's region from a body surface, and controlling volumes of the blood vessels at a fixed level. The measurement examinee's region suited to this measurement method involves, generally, using a finger of hand. The sphygmomanometer using the volume compensation method so far employs a finger cuff of such a type as to be wound around the finger, and a finger cuff of such a type as to insert the finger into a cylindrical cuff. Note that the following Patent documents exist as technologies pertaining to the present application.

[Patent document 1] Japanese Laid-open Patent Publication No. 09-289977
[Patent document 2] Japanese National Publication of International Patent Application No. 2013-510642
[Patent document 3] Japanese Laid-open Patent Publication No. 61-238227
[Patent document 4] Japanese Unexamined Utility Model Application Publication No. 02-10805
[Patent document 5] Japanese Laid-open Patent Publication No. 2005-40498

### SUMMARY

The finger is adopted as the measurement examinee's region, in which case the blood pressure of the artery of the finger is measured by winding the cuff around the finger. On the occasion of measuring the blood pressure of the artery of the finger by winding the cuff around the finger and when measuring the blood pressure in such a state that an entire periphery of the finger is covered with the cuff, a vein of the finger is excessively compressed, and the finger gets congested with the blood in some cases. The blood pressures of the artery of the finger are consecutively measured by continuously controlling the compression and decompression of the finger. When consecutively measuring the blood pressures of the artery of the finger, the finger gets congested with the blood, resulting in an apprehension of adversely affecting measurement results. It is an object of the present application, which is contrived in view of the problems described above, to provide a technology of restraining a finger from being congested with blood when measuring a blood pressure.

According to an aspect of the application, a sphygmomanometer cuff for finger, includes: a base unit including a first curved portion extending along a part of periphery of a finger defined as a measurement examinee's target region; at least one movable unit connected via a rotatable unit to the base unit, including a second curved portion extending along another part of periphery of the finger, and being attachable to the finger in a state of an opening being formed along the periphery of the finger; an adjustment unit configured to adjust an angle of the movable unit to the base unit; and a compression unit provided along at least a part of an inner peripheral portion formed along the periphery of the finger by the base unit, the movable unit and the opening, and compressing the part of periphery of the finger.

According to the present application, it is feasible to restrain a measurement examinee's region from being congested with the blood when measuring the blood pressure.

### BRIEF DESCRIPTION OF DRAWINGS

FIG. 1 is a diagram illustrating one example of a blood pressure measuring apparatus according to a first embodiment.

FIG. 2 is a diagram illustrating one example of a cuff according to the first embodiment.

FIG. 3 is a diagram illustrating one example of the cuff according to the first embodiment.

FIG. 4 is a diagram illustrating one example of the cuff according to the first embodiment.

FIGS. 5A and 5B are diagrams each illustrating one example of the cuff according to the first embodiment.

FIG. 6 is a diagram illustrating one example of the cuff according to the first embodiment.

FIG. 7 is a diagram illustrating one example of the cuff according to the first embodiment.

FIGS. 8A and 8B are diagrams each illustrating one example of the cuff according to the first embodiment.

FIGS. 9A and 9B are diagrams each illustrating one example of the cuff according to the first embodiment.

FIG. 10 is a diagram illustrating one example of the cuff according to a second embodiment.

FIGS. 11A and 11B are diagrams each illustrating one example of the cuff according to the second embodiment.

FIG. 12 is a diagram illustrating one example of the cuff according to the second embodiment.

FIGS. 13A and 13B are diagrams each illustrating one example of the cuff according to the second embodiment.

FIGS. 14A and 14B are diagrams each illustrating one example of the cuff according to the third embodiment.

FIGS. 15A and 15B are diagrams each illustrating one example of the cuff according to the third embodiment.

FIG. 16 is a diagram illustrating one example of the cuff according to the third embodiment.

FIGS. 17A and 17B are diagrams each illustrating one example of the cuff according to the third embodiment.

FIGS. 18A and 18B are diagrams each illustrating one example of the cuff according to the third embodiment.

### DESCRIPTION OF EMBODIMENT

Embodiments of the present application will hereinafter be described based on the drawings. Note that the embodiments to be described below will each demonstrate one example of carrying out the present application, but do not limit specific configurations for describing the present application as follows.

### First Embodiment

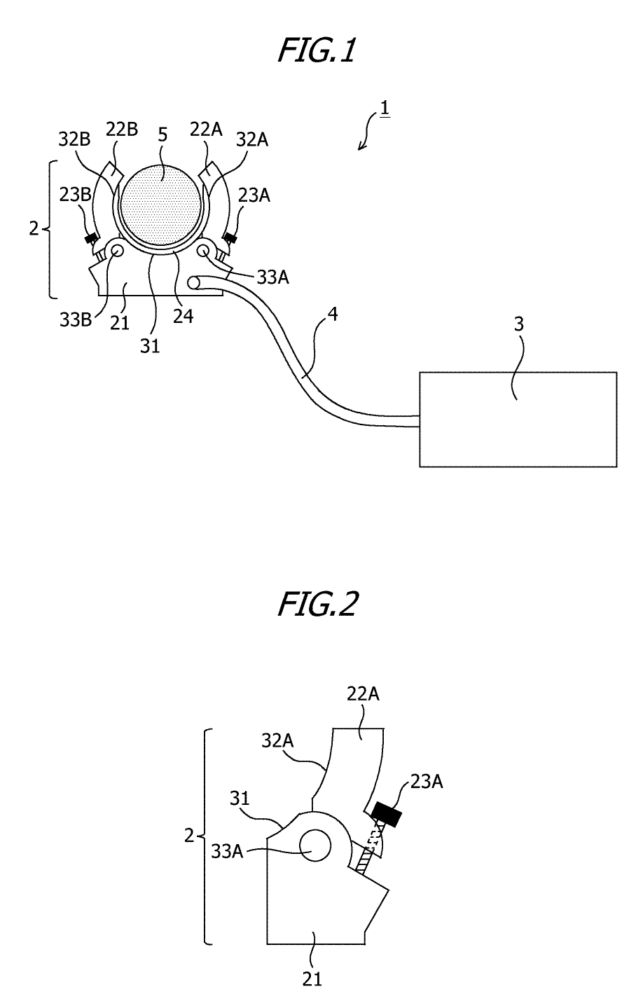

A first embodiment will be described with reference to FIGS. 1 through 9. FIG. 1 is a diagram illustrating one example of a blood pressure measuring apparatus 1 according to the first embodiment. The blood pressure measuring apparatus 1 includes a cuff 2 wound around a finger 5 of a measurement examinee, a sphygmomanometer body 3 that consecutively measures blood pressures of an artery of the finger 5 via a cuff 2, and a cable 4 that connects the cuff 2 and the sphygmomanometer body 3 together. The blood pressure measuring apparatus 1 may consecutively measure the blood pressures of the artery of the finger 5 by using, e.g., a volume compensation method, and may also consecutively measure the blood pressures of the artery of the finger 5 by other methods. The cuff 2 is one example of a “sphygmomanometer cuff for finger”. The cuff 2 is attached to the finger 5 defined as a measurement examinee's target region. The cuff 2 includes a base unit 21, movable units 22A, 22B, adjustment screws 23A, 23B, an airbag 24, and rotatable units 33A, 33B. The cuff 2 further includes a detection unit, within the base unit 21, for detecting pressure pulse waves of the artery of the finger 5.

The sphygmomanometer body 3 includes a supply device that supplies air to the airbag 24, and a control device that controls actuation of the cuff 2. The control device of the sphygmomanometer body 3 includes a CPU (Central Processing Unit) for executing a variety of arithmetic processes, and a memory or another equivalent storage for storing a program and a various items of data. The memory is exemplified by a ROM (Read Only Memory) and a RAM (Random Access Memory). The control device of the sphygmomanometer body 3 controls a pressure and a supply quantity of the air supplied to the airbag 24, and acquires the various items of data detected by the cuff 2. One cable 4 may be available, and a plurality of cables 4 may also be available.

The base unit 21 has a curved portion 31 extending along a part of periphery of the finger 5. The movable unit 22A has a curved portion 32A extending along a part of periphery of the finger 5. The movable unit 22B has a curved portion 32B extending along a part of periphery of the finger 5. The movable unit 22A is connected to the base unit 21 via the rotatable unit 33A, and the movable unit 22B is connected to the base unit 21 via the rotatable unit 33B. The movable units 22A, 22B are disposed so that the curved portion 32A of the movable unit 22A has a face-to-face relationship with the curved portion 32B of the movable unit 22B.

FIG. 2 is a diagram illustrating one example of the cuff 2 according to the first embodiment. The adjustment screw 23A adjusts an angle (θ) of the movable unit 22A to the base unit 21. The adjustment screw 23A is one example of an “adjustment unit”. The adjustment screw 23A is connected to the base unit 21 and to the movable unit 22A. The adjustment screw 23A penetrates a proximal end of the movable unit 22A, and a front end of the adjustment screw 23A is embedded into the base unit 21. A force is applied to the movable unit 22A by rotating the adjustment screw 23A, and the rotatable unit 33A rotates, whereby the angle of the movable unit 22A to the base unit 21 varies. For example, the force is applied to the movable unit 22A in such a direction as to get away from the base unit 21 by fastening the adjustment screw 23A, and the angle of the movable unit 22A to the base unit 21 decreases upon a rotation of the rotatable unit 33A. For instance, the force is applied to the movable unit 22A in such a direction as get close to the base unit 21 by unfastening the adjustment screw 23A, and the rotatable unit 33A rotes, whereby the angle of the movable unit 22A to the base unit 21 increases.

The adjustment screw 23B adjusts the angle (θ) of the movable unit 22B to the base unit 21. The adjustment screw 23B is one example of the “adjustment unit”. The adjustment screw 23B is connected to the base unit 21 and to the movable unit 22B. The adjustment screw 23B penetrates a proximal end of the movable unit 22B, and a front end of the adjustment screw 23B is embedded into the base unit 21. A force is applied to the movable unit 22B by rotating the adjustment screw 23B, and the rotatable unit 33B rotates, whereby the angle of the movable unit 22B to the base unit 21 varies. For example, the force is applied to the movable unit 22B in such a direction as to get away from the base unit 21 by fastening the adjustment screw 23B, and the angle of the movable unit 22B to the base unit 21 decreases upon the rotation of the rotatable unit 33B. For instance, the force is applied to the movable unit 22B in such a direction as get close to the base unit 21 by unfastening the adjustment screw 23B, and the rotatable unit 33B rotes, whereby the angle of the movable unit 22B to the base unit 21 increases.

When attaching the cuff 2 to the finger 5, the angles of the movable units 22A, 22B to the base unit 21 increase by unfastening the adjustment screws 23A, 23B, thereby reaching an open state of the cuff 2. In the open state of the cuff 2, the finger 5 is received inside of the cuff 2, and the angles of the movable units 22A, 22B to the base unit 21 decrease by fastening the adjustment screws 23A, 23B. The finger 5 is received inside of the cuff 2, and the angles of the movable units 22A, 22B to the base unit 21 decrease, thereby fixing the cuff 2 to the finger 5. Accordingly, the adjustment screws 23A, 23B are fastened, thereby fixing the cuff 2 to the finger 5 in such a state that the base unit 21 and the movable units 22A, 22B are attached to the periphery of the finger 5.

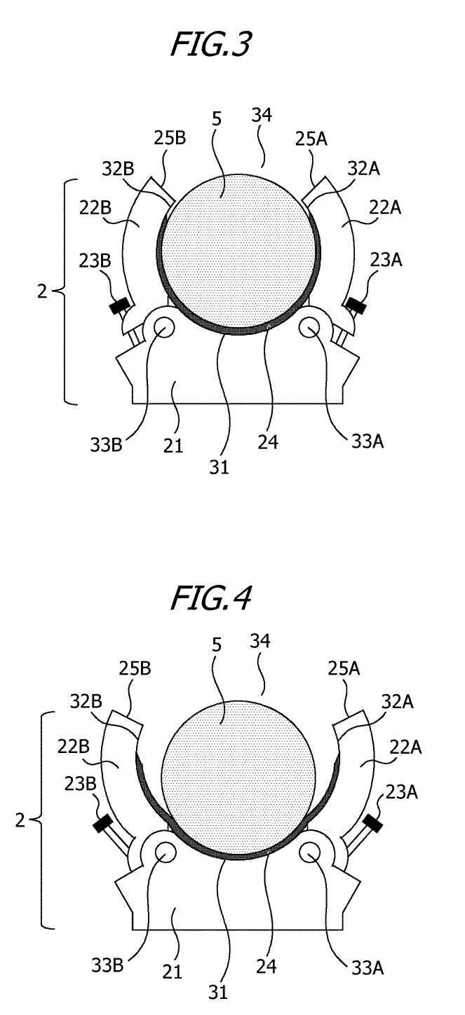

FIG. 3 is a diagram illustrating one example of the cuff 2 according to the first embodiment. FIG. 3 depicts a state in which the cuff 2 is attached to and fixed to the finger 5 by fastening the adjustment screws 23A, 23B. A front end 25A of the movable unit 22A is not brought into contact with a front end 25B of the movable unit 22B, and an opening 34 is formed between the front end 25A of the movable unit 22A and the front end 25B of the movable unit 22B. Accordingly, the base unit 21 and the movable units 22A, 22B are attached to the finger 5 in a state of the opening 34 being formed along a part of periphery of the finger 5. When the cuff 2 is fixed to the finger 5, the front end 25A of the movable unit 22A and the front end 25B of the movable unit 22B are distanced at about 5 mm, and the distance therebetween may, however, take other values without being limited to 5 mm.

In the cuff 2 illustrated in FIG. 3, the movable units 22A, 22B are symmetric in shape with respect to an axis of the finger 5 (bilateral symmetry on the sheet surface of FIG. 3). The curved portion 32A of the movable unit 22A and the curved portion 32B of the movable unit 22B are symmetric in shape with respect to the axis of the finger 5. A length of the curved portion 32A of the movable unit 22A is therefore the same as a length of the curved portion 32B of the movable unit 22B.

The airbag 24 expands upon being supplied with the air from the sphygmomanometer body 3 via the cable 4. The airbag 24 is one example of a “compression unit”. The airbag 24 is provided along at least a part of inner peripheral portion of the cuff 2, which is formed by the base unit 21, the movable units 22A, 22B and the opening 34. The expansion of the airbag 24 compresses a part of periphery of the finger 5. An air pressure of and an air supply quantity to the airbag 24 are controlled, thereby compressing and decompressing the finger 5. The finger 5 is compressed and decompressed alternately in a repetitive manner, whereby the blood pressures of the artery of the finger 5 are consecutively measured. The finger 5 is fixed to the cuff 2 by the adjustment screws 23A, 23B, and hence, when the airbag 24 expands to compress the part of periphery of the finger 5, the cuff 2 is restrained from coming off the finger 5. FIG. 4 is a diagram illustrating one example of the cuff 2 according to the first embodiment. FIG. 4 depicts an open state of the cuff 2. The cuff 2 is detached from the finger 5 in the open state of the cuff 2.

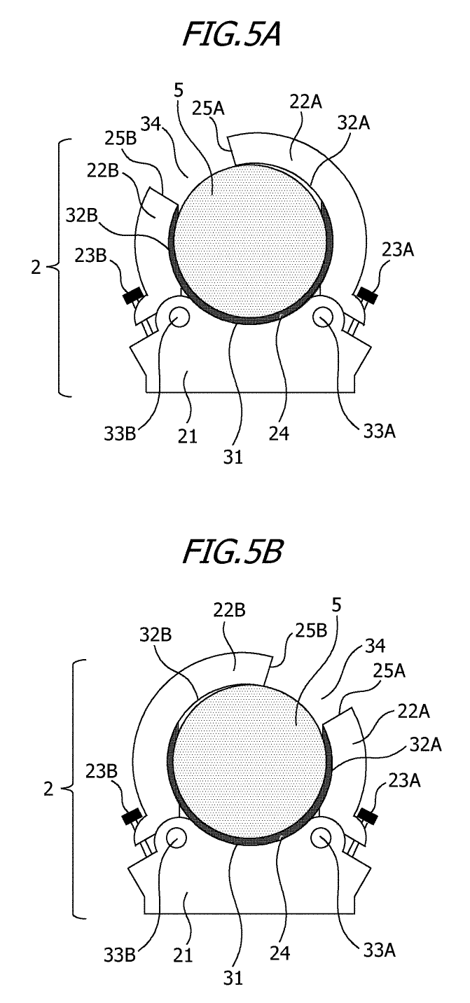

FIGS. 5A and 5B are diagrams each illustrating one example of the cuff 2 according to the first embodiment. In the cuff 2 illustrated in FIG. 5A, the movable units 22A, 22B are asymmetric in shape with respect to the axis of the finger 5 (bilateral asymmetry on the sheet surface of FIG. 5A). The curved portion 32A of the movable unit 22A and the curved portion 32B of the movable unit 22B are asymmetric in shape with respect to the axis of the finger 5, and the length of the curved portion 32A of the movable unit 22A is larger than the length of the curved portion 32B of the movable unit 22B. The length of the curved portion 32A of the movable unit 22A is larger than the length of the curved portion 32B of the movable unit 22B, and therefore, when the airbag 24 expands to compress the part of periphery of the finger 5, the cuff 2 is further restrained from coming off the finger 5. Accordingly, it is feasible to surely fix the cuff 2 to the finger 5.

In the cuff 2 illustrated in FIG. 5B, the movable units 22A, 22B are asymmetric in shape with respect to the axis of the finger 5 (bilateral asymmetry on the sheet surface of FIG. 5B). The curved portion 32A of the movable unit 22A and the curved portion 32B of the movable unit 22B are asymmetric in shape with respect to the axis of the finger 5, and the length of the curved portion 32B of the movable unit 22B is larger than the length of the curved portion 32A of the movable unit 22A. The length of the curved portion 32B of the movable unit 22B is larger than the length of the curved portion 32A of the movable unit 22A, and hence, when the airbag 24 expands to compress the part of periphery of the finger 5, the cuff 2 is still further restrained from coming off the finger 5. It is therefore feasible to more surely fix the cuff 2 to the finger 5.

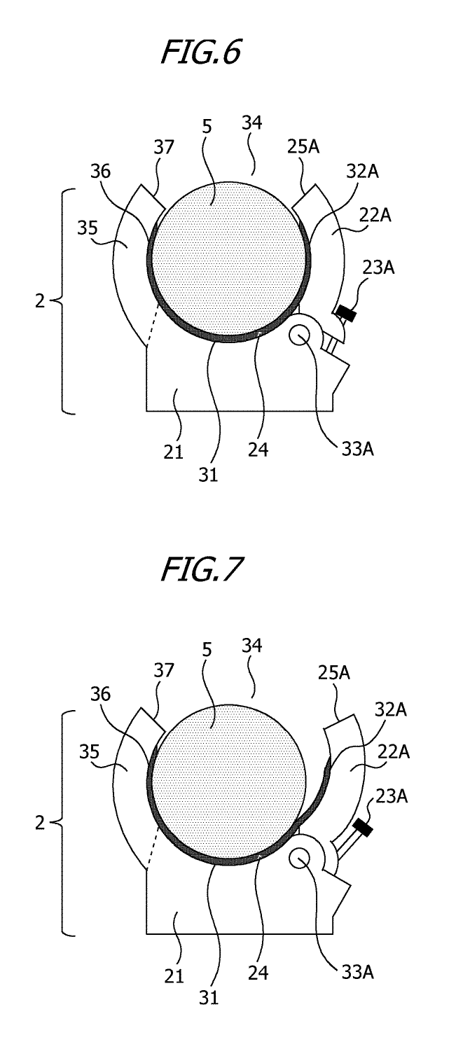

The example of the configuration of the cuff 2 including one base unit 21, the plurality of movable units 22 (22A, 22B), the plurality of adjustment screws 23 (23A, 23B) and the plurality of rotatable units 33 (33A, 33B), has been described with reference to FIGS. 1 through 5. Described with reference to FIGS. 6 through 8 is an example of a configuration of the cuff 2 including one base unit 21, one movable unit 22 (22A), one adjustment screw 23 (23A) and one rotatable unit 33 (33A). FIG. 6 is a diagram illustrating one example of the cuff 2 according to the first embodiment. As illustrated in FIG. 6, the base unit 21 is provided with a protruded portion 35 taking the same shape as the shape of the movable unit 22B depicted in FIG. 3. In other words, the base unit 21 and the protruded portion 35 are integrally configured. The protruded portion 35 has a curved portion 36 extending along a part of periphery of the finger 5. The movable unit 22A and the protruded portion 35 are disposed so that the curved portion 32A of the movable unit 22A has a face-to-face relationship with the curved portion 36 of the protruded portion 35.

FIG. 6 depicts a state in which the cuff 2 is attached and fixed to the finger 5 by fastening the adjustment screw 23A. The front end 25A of the movable unit 22A is not brought into contact with a front end 37 of the protruded portion 35, and the opening 34 is formed between the front end 25A of the movable unit 22A and the front end 37 of the protruded portion 35. Accordingly, the base unit 21, the protruded portion 35 and the movable unit 22A are attached to the finger 5 in the state of the opening 34 being formed along the part of periphery of the finger 5. In the cuff 2 illustrated in FIG. 6, the curved portion 32A of the movable unit 22A and the curved portion 36 of the protruded portion 35 are symmetric in shape with respect to the axis of the finger 5 (bilateral symmetry on the sheet surface of FIG. 6), and the length of the curved portion 32A of the movable unit 22A is the same as a length of the curved portion 36 of the protruded portion 35.

The airbag 24 is provided along at least the part of inner peripheral portion of the cuff 2, which is formed by the base unit 21, the movable unit 22A, the opening 34 and the protruded portion 35. The expansion of the airbag 24 compresses the part of periphery of the finger 5. The cuff 2 is fixed to the finger 5 by the adjustment screw 23A, and hence, when the airbag 24 expands to compress the part of periphery of the finger 5, the cuff 2 is restrained from coming off the finger 5. FIG. 7 is a diagram illustrating one example of the cuff 2 according to the first embodiment. FIG. 7 depicts an open state of the cuff 2. The cuff 2 is attached to and detached from the finger 5 in the open state of the cuff 2.

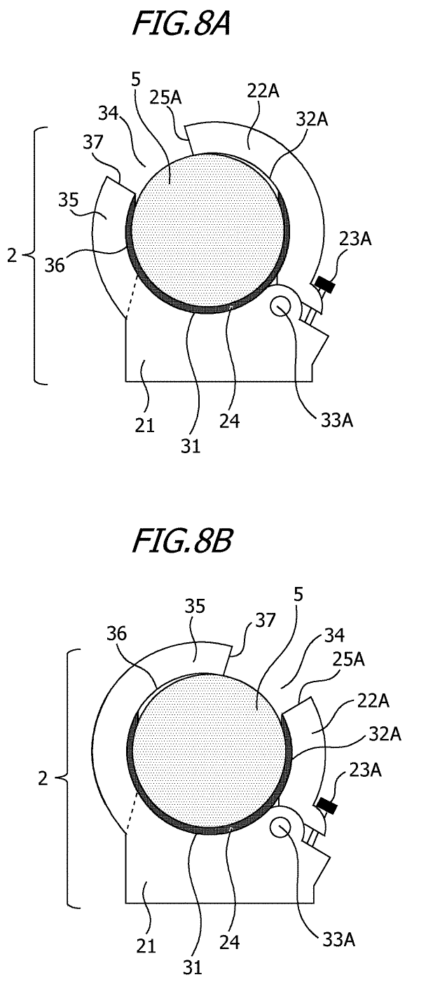

FIGS. 8A and 8B are diagrams each illustrating one example of the cuff 2 according to the first embodiment. In the cuff 2 depicted in FIG. 8A, the curved portion 32A of the movable unit 22A and the curved portion 36 of the protruded portion 35 are asymmetric in shape with respect to the axis of the finger 5 (bilateral asymmetry on the sheet surface of FIG. 8A), and the length of the curved portion 32A of the movable unit 22A is larger than the length of the curved portion 36 of the protruded portion 35. The length of the curved portion 32A of the movable unit 22A is larger than the length of the curved portion 36 of the protruded portion 35, and hence, when the airbag 24 expands to compress the part of periphery of the finger 5, the cuff 2 is further restrained from coming off the finger 5. It is therefore feasible to more surely fix the cuff 2 to the finger 5.

In the cuff 2 depicted in FIG. 8B, the curved portion 32A of the movable unit 22A and the curved portion 36 of the protruded portion 35 are asymmetric in shape with respect to the axis of the finger 5 (bilateral asymmetry on the sheet surface of FIG. 8B), and the length of the curved portion 36 of the protruded portion 35 is larger than the length of the curved portion 32A of the movable unit 22A. The length of the curved portion 36 of the protruded portion 35 is larger than the length of the curved portion 32A of the movable unit 22A, and consequently, when the airbag 24 expands to compress the part of periphery of the finger 5, the cuff 2 is still further restrained from coming off the finger 5. Accordingly, it is possible to much more surely fix the cuff 2 to the finger 5.

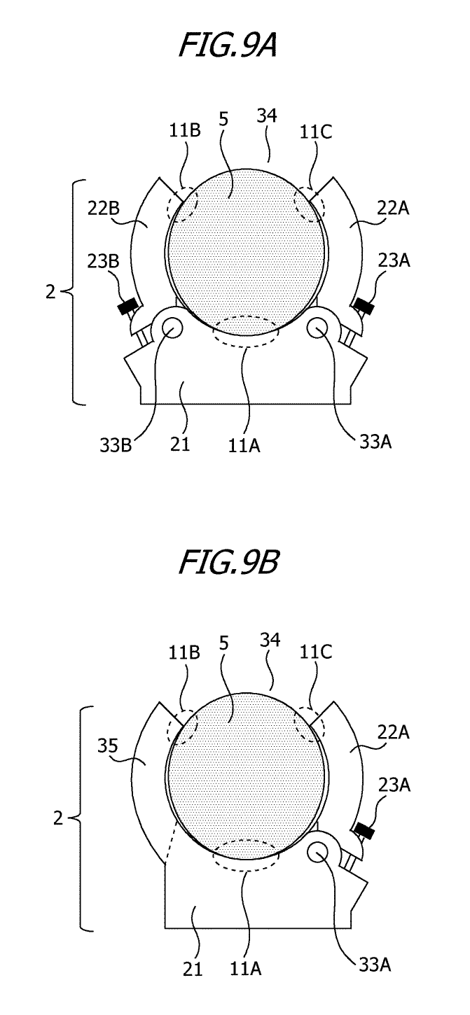

FIGS. 9A and 9B are diagrams each illustrating one example of the cuff 2 according to the first embodiment. As depicted in FIGS. 9A and 9B, the cuff 2 may support the finger 5 at three points (e.g., areas circumscribed dotted lines 11A, 11B, 11C). FIG. 9A illustrates one example of the cuff 2 including one base unit 21, the plurality of movable units 22 (22A, 22B), the plurality of adjustment screws 23 (23A, 23B) and the plurality of rotatable units 33 (33A, 33B). In the example of the configuration of the cuff 2 depicted in FIG. 9A, the finger 5 is supported by a predetermined portion of the base unit 21, a predetermined portion of the movable unit 22A, and a predetermined portion of the movable unit 22B. FIG. 9B illustrates one example of the cuff 2 including one base unit 21, one movable unit 22 (22A), one adjustment screw 23 (23A) and one rotatable unit 33 (33A). In the example of the configuration of the cuff 2 depicted in FIG. 9B, the finger 5 is supported by a predetermined portion of the base unit 21, a predetermined portion of the protruded portion 35 and a predetermined portion of the movable unit 22A. Note that the cuff 2 may support the finger 5 at three points via the airbag 24, in FIGS. 9A and 9B, though the illustration of the airbag 24 is omitted.

### Second Embodiment

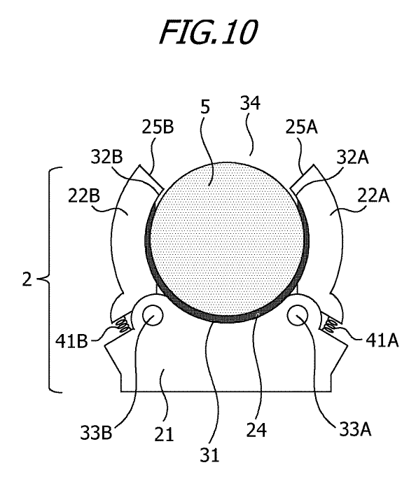

A second embodiment will be described with reference to FIGS. 10 through 13. The sphygmomanometer body 3 and the cable 4, which are equipped in the blood pressure measuring apparatus 1 according to the second embodiment, are the same as those in the first embodiment, and their repetitive explanations are omitted. The same components as those in the first embodiment are marked with the same numerals and symbols as those in the first embodiment, and their repetitive explanations are omitted. FIG. 10 is a diagram illustrating one example of the cuff 2 according to the second embodiment. The cuff 2 includes the base unit 21, the movable units 22A, 22B, the airbag 24, the rotatable units 33A, 33B, and the adjustment springs 41A, 41B. FIG. 10 depicts a state in which the cuff 2 is attached and fixed to the finger 5.

The adjustment spring 41A adjusts an angle (θ) of the movable unit 22A to the base unit 21. The adjustment spring 41A is one example of an “adjustment unit”. The adjustment spring 41A is one example of an “adjustment elastic body”. The adjustment spring 41A is instanced by a coil spring and a plate spring. As depicted in FIG. 10, the adjustment spring 41A is disposed between the base unit 21 and the proximal end of the movable unit 22A, and is connected to the base unit 21 and the proximal end of the movable unit 22A. When the adjustment spring 41A stretches, a force is applied to the movable unit 22A in such a direction as to get away from the base unit 21, and the rotatable unit 33A rotates to thereby decrease the angle of the movable unit 22A to the base unit 21. When the adjustment spring 41A shrinks, the force is applied to the movable unit 22A in such a direction as to get close to the base unit 21, and the rotatable unit 33A rotates to thereby increase the angle of the movable unit 22A to the base unit 21.

The adjustment spring 41B adjusts the angle (θ) of the movable unit 22B to the base unit 21. The adjustment spring 41B is one example of the “adjustment unit”. The adjustment spring 41B is one example of the “adjustment elastic body”. The adjustment spring 41B is instanced by the coil spring and the plate spring. As illustrated in FIG. 10, the adjustment spring 41B is disposed between the base unit 21 and the proximal end of the movable unit 22B, and is connected to the base unit 21 and the proximal end of the movable unit 22B. When the adjustment spring 41B stretches, the force is applied to the movable unit 22B in such a direction as to get away from the base unit 21, and the rotatable unit 33B rotates to thereby decrease the angle of the movable unit 22B to the base unit 21. When the adjustment spring 41B shrinks, the force is applied to the movable unit 22B in such a direction as to get close to the base unit 21, and the rotatable unit 33B rotates to thereby increase the angle of the movable unit 22B to the base unit 21.

When attaching the cuff 2 to the finger 5, the finger 5 is inserted into a gap between the movable units 22A, 22B, and the angles of the movable units 22A, 22B to the base unit 21 are consequently increased, thereby leading to the open state of the cuff 2. When the finger 5 is received inside of the cuff 2, the angles of movable units 22A, 22B to the base unit 21 are decreased. The finger 5 is received inside of the cuff 2, and the angles of movable units 22A, 22B to the base unit 21 are decreased, whereby the cuff 2 is fixed to the finger 5. Accordingly, the cuff 2 is fixed to the finger 5 in a state in which the base unit 21 and the movable units 22A, 22B are attached to the periphery of the finger 5.

The front end 25A of the movable unit 22A is not brought into contact with the front end 25B of the movable unit 22B, and the opening 34 is formed between the front end 25A of the movable unit 22A and the front end 25B of the movable unit 22B. Accordingly, the base unit 21 and the movable units 22A, 22B are attached to the finger 5 in the state of the opening 34 being formed along a part of periphery of the finger 5. In the cuff 2 illustrated in FIG. 10, the movable units 22A, 22B are symmetric in shape with respect to the axis of the finger 5 (the bilateral symmetry on the sheet surface of FIG. 10). Therefore, the curved portion 32A of the movable unit 22A and the curved portion 32B of the movable unit 22B are symmetric in shape with respect to the axis of the finger 5. Consequently, the length of the curved portion 32A of the movable unit 22A is the same as the length of the curved portion 32B of the movable unit 22B.

The airbag 24 is provided along at least a part of inner peripheral portion of the cuff 2, which is formed by the base unit 21, the movable units 22A, 22B and the opening 34. The expansion of the airbag 24 compresses a part of periphery of the finger 5. The cuff 2 is fixed to the finger 5 by the adjustment springs 41A, 41B, and hence, when the airbag 24 expands to compress the part of periphery of the finger 5, the cuff 2 is restrained from coming off the finger 5.

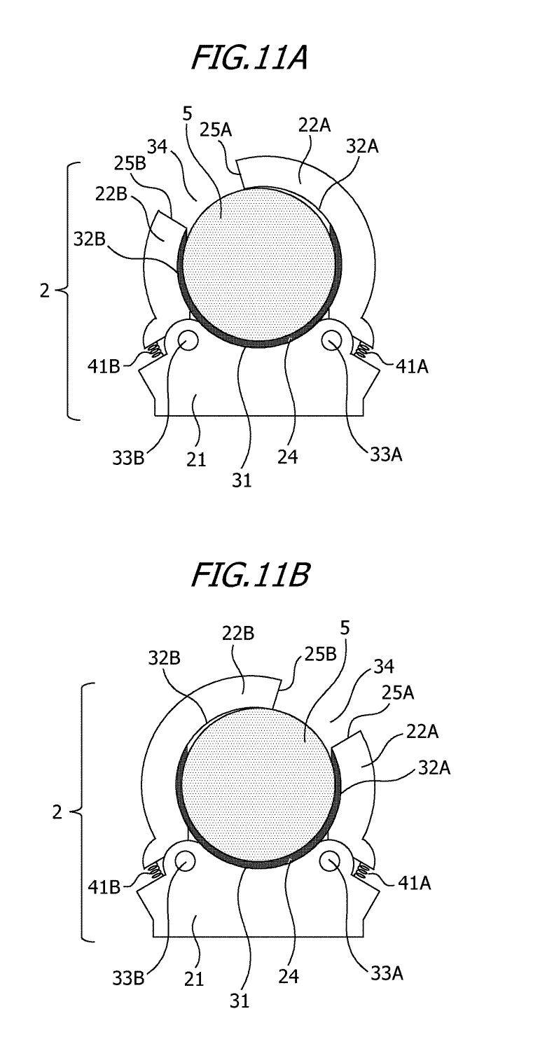

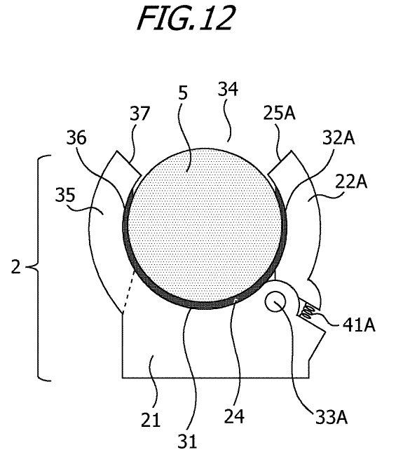

FIGS. 11A and 11B are diagrams each illustrating one example of the cuff 2 according to the second embodiment. In the cuff 2 depicted in FIG. 11A, the movable units 22A, 22B are asymmetric in shape with respect to the axis of the finger 5 (bilateral asymmetry on the sheet surface of FIG. 11A). The curved portion 32A of the movable unit 22A and the curved portion 32B of the movable unit 22B are asymmetric in shape with respect to the axis of the finger 5, and the length of the curved portion 32A of the movable unit 22A is larger than the length of the curved portion 32B of the movable unit 22B. The length of the curved portion 32A of the movable unit 22A is larger than the length of the curved portion 32B of the movable unit 22B, and therefore, when the airbag 24 expands to compress the part of periphery of the finger 5, the cuff 2 is further restrained from coming off the finger 5. Accordingly, it is feasible to more surely fix the cuff 2 to the finger 5.

In the cuff 2 depicted in FIG. 11B, the movable units 22A, 22B are asymmetric in shape with respect to the axis of the finger 5 (bilateral asymmetry on the sheet surface of FIG. 11B). The curved portion 32A of the movable unit 22A and the curved portion 32B of the movable unit 22B are asymmetric in shape with respect to the axis of the finger 5, and the length of the curved portion 32B of the movable unit 22B is larger than the length of the curved portion 32A of the movable unit 22A. The length of the curved portion 32B of the movable unit 22B is larger than the length of the curved portion 32A of the movable unit 22A, and hence, when the airbag 24 expands to compress the part of periphery of the finger 5, the cuff 2 is still further restrained from coming off the finger 5. It is therefore possible to more surely fix the cuff 2 to the finger 5.

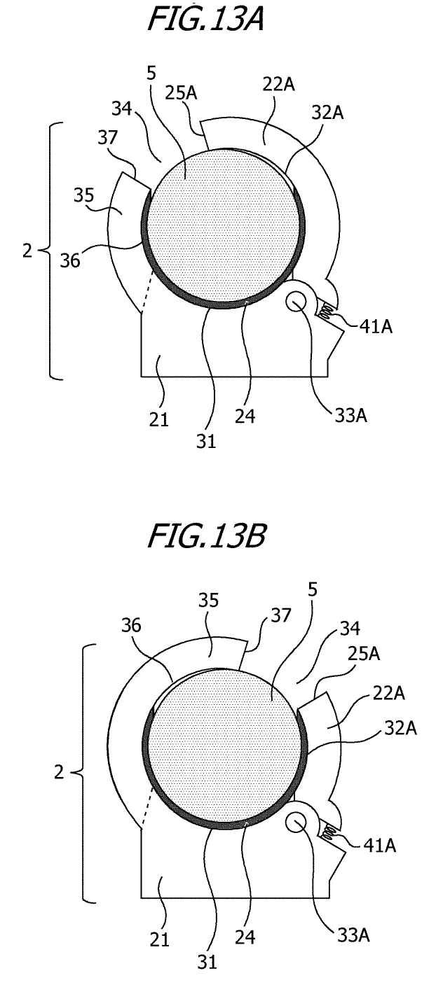

The example of the configuration of the cuff 2 including one base unit 21, the plurality of movable units 22 (22A, 22B), the plurality of rotatable units 33 (33A, 33B) and the plurality of adjustment springs 41 (41A, 41B), has been described with reference to FIGS. 10 and 11. Described with reference to FIGS. 12 and 13 is an example of a configuration of the cuff 2 including one base unit 21, one movable unit 22 (22A), one rotatable unit 33 (33A) and one adjustment spring 41 (41A). FIG. 12 is a diagram illustrating one example of the cuff 2 according to the second embodiment. As illustrated in FIG. 12, the base unit 21 is provided with the protruded portion 35 taking the same shape as the shape of the movable unit 22B depicted in FIG. 10. In other words, the base unit 21 and the protruded portion 35 are integrally configured. The protruded portion 35 has the curved portion 36 extending along a part of periphery of the finger 5. The movable unit 22A and the protruded portion 35 are disposed so that the curved portion 32A of the movable unit 22A has the face-to-face relationship with the curved portion 36 of the protruded portion 35.

FIG. 12 depicts a state in which the cuff 2 is attached and fixed to the finger 5. The front end 25A of the movable unit 22A is not brought into contact with the front end 37 of the protruded portion 35, and the opening 34 is formed between the front end 25A of the movable unit 22A and the front end 37 of the protruded portion 35. Accordingly, the base unit 21, the protruded portion 35 and the movable unit 22A are attached to the finger 5 in the state of the opening 34 being formed along the part of periphery of the finger 5. In the cuff 2 illustrated in FIG. 12, the curved portion 32A of the movable unit 22A and the curved portion 36 of the protruded portion 35 are symmetric in shape with respect to the axis of the finger 5 (bilateral symmetry on the sheet surface of FIG. 12), and the length of the curved portion 32A of the movable unit 22A is the same as the length of the curved portion 36 of the protruded portion 35.

The airbag 24 is provided along at least the part of inner peripheral portion of the cuff 2, which is formed by the base unit 21, the movable unit 22A, the opening 34 and the protruded portion 35. The expansion of the airbag 24 compresses the part of periphery of the finger 5. The cuff 2 is fixed to the finger 5 by the adjustment spring 41A, and hence, when the airbag 24 expands to compress the part of periphery of the finger 5, the cuff 2 is restrained from coming off the finger 5.

FIGS. 13A and 13B are diagrams each illustrating one example of the cuff 2 according to the second embodiment. In the cuff 2 depicted in FIG. 13A, the curved portion 32A of the movable unit 22A and the curved portion 36 of the protruded portion 35 are asymmetric in shape with respect to the axis of the finger 5 (bilateral asymmetry on the sheet surface of FIG. 13A), and the length of the curved portion 32A of the movable unit 22A is larger than the length of the curved portion 36 of the protruded portion 35. The length of the curved portion 32A of the movable unit 22A is larger than the length of the curved portion 36 of the protruded portion 35, and hence, when the airbag 24 expands to compress the part of periphery of the finger 5, the cuff 2 is further restrained from coming off the finger 5. It is therefore possible to more surely fix the cuff 2 to the finger 5.

In the cuff 2 depicted in FIG. 13B, the curved portion 32A of the movable unit 22A and the curved portion 36 of the protruded portion 35 are asymmetric in shape with respect to the axis of the finger 5 (bilateral asymmetry on the sheet surface of FIG. 13B), and the length of the curved portion 36 of the protruded portion 35 is larger than the length of the curved portion 32A of the movable unit 22A. The length of the curved portion 36 of the protruded portion 35 is larger than the length of the curved portion 32A of the movable unit 22A, and consequently, when the airbag 24 expands to compress the part of periphery of the finger 5, the cuff 2 is still further restrained from coming off the finger 5. It is therefore possible to much more surely fix the cuff 2 to the finger 5.

### Third Embodiment

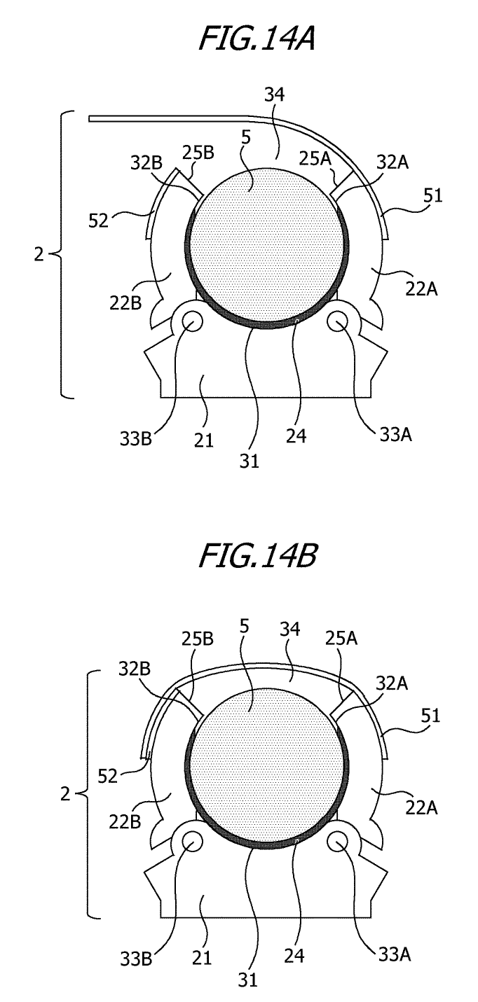

A third embodiment will be described with reference to FIGS. 14 through 18. The sphygmomanometer body 3 and the cable 4, which are equipped in the blood pressure measuring apparatus 1 according to the third embodiment, are the same as those in the first embodiment, and their repetitive explanations are omitted. The same components as those in the first embodiment are marked with the same numerals and symbols as those in the first embodiment, and their repetitive explanations are omitted. FIGS. 14A and 14B are diagrams each illustrating one example of the cuff 2 according to the third embodiment. The cuff 2 includes the base unit 21, the movable units 22A, 22B, the airbag 24, the rotatable units 33A, 33B, and adjustment band members 51, 52. FIG. 14A depicts a state in which the cuff 2 is attached to the finger 5. FIG. 14B depicts a state in which the cuff 2 is attached and fixed to the finger 5.

The adjustment band member 51 adjusts the angle (θ) of the movable unit 22A to the base unit 21. The adjustment band member 52 adjusts the angle (θ) of the movable unit 22B to the base unit 21. The adjustment band members 51, 52 are each one example of the “adjustment unit”. The adjustment band members 51, 52 are each instanced by an adjustment belt or a hook and loop fastener. As depicted in FIGS. 14A and 14B, one end of the adjustment band member 51 is provided along an outer peripheral surface of the movable unit 22A, and the adjustment band member 52 is provided along an outer peripheral surface of the movable unit 22B. A length of the adjustment band member 51 is larger than a length of the adjustment band member 52. The force is applied to the movable unit 22A in such a direction as to get away from the base unit 21 by pulling the adjustment band member 51, and the rotatable unit 33A rotates to change the angle of the movable unit 22A to the base unit 21. The force is applied to the movable unit 22B in such a direction as to get away from the base unit 21 by pulling the adjustment band member 52, and the rotatable unit 33B rotates to change the angle of the movable unit 22B to the base unit 21.

The other end of the adjustment band member 51 is detachably fixed to the adjustment band member 52. An arbitrary position of the other end of the adjustment band member 51 is attachable to the adjustment band member 52. When attaching the cuff 2 to the finger 5, the other end of the adjustment band member 51 is detached from the adjustment band member 52, whereby the cuff 2 reaches the open state. The finger 5 is received inside of the cuff 2 in the open state of the cuff 2. The other end of the adjustment band member 51 is attached to the adjustment band member 52 while pulling the adjustment band members 51, 52, thereby decreasing the angles of the movable units 22A, 22B to the base unit 21. The finger 5 is received inside of the cuff 2, and the angles of the movable units 22A, 22B to the base unit 21 are decreased, whereby the cuff 2 is fixed to the finger 5. Accordingly, the cuff 2 is fixed to the finger 5 in such a state that the base unit 21 and the movable units 22A, 22B are attached to the periphery of the finger 5.

The front end 25A of the movable unit 22A is not brought into contact with the front end 25B of the movable unit 22B, and the opening 34 is formed between the front end 25A of the movable unit 22A and the front end 25B of the movable unit 22B. Accordingly, the movable units 22A, 22B are attached to the finger 5 in the state of the opening 34 being formed along the part of periphery of the finger 5. As illustrated in FIG. 14B, the other end of the adjustment band member 51 is attached to the adjustment band member 52, in which case the opening 34 is covered with a part of the adjustment band member 51. In the cuff 2 illustrated in FIGS. 14A and 14B the movable units 22A, 22B are symmetric in shape with respect to the axis of the finger 5 (bilateral symmetry on the sheet surface of FIGS. 14A and 14B). The curved portion 32A of the movable unit 22A and the curved portion 32B of the movable unit 22B are therefore symmetric in shape with respect to the axis of the finger 5. Accordingly, the length of the curved portion 32A of the movable unit 22A is the same as the length of the curved portion 32B of the movable unit 22B.

The airbag 24 is provided along at least the part of inner peripheral portion of the cuff 2, which is formed by the base unit 21, the movable units 22A, 22B and the opening 34. The expansion of the airbag 24 compresses the part of periphery of the finger 5. The cuff 2 is fixed to the finger 5 by the adjustment band members 51, 52, and hence, when the airbag 24 expands to compress the part of periphery of the finger 5, the cuff 2 is restrained from coming off the finger 5.

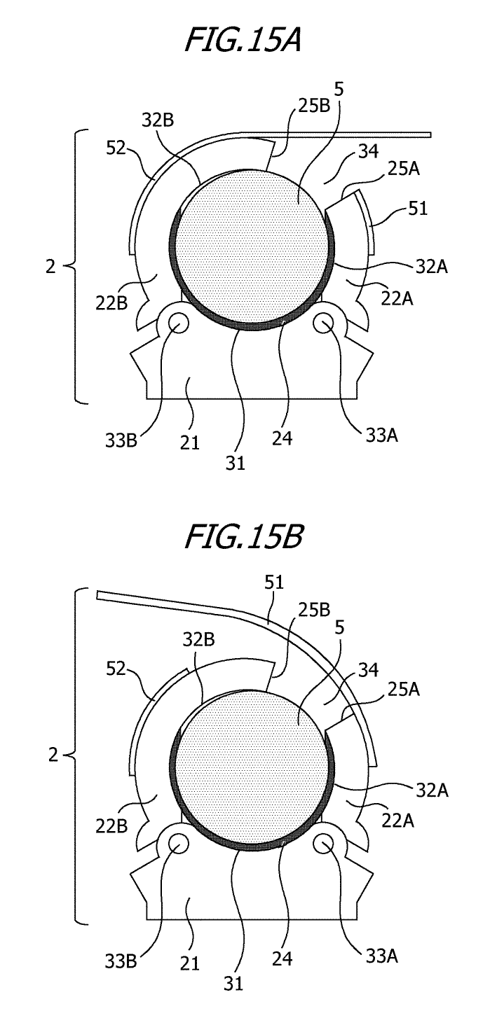

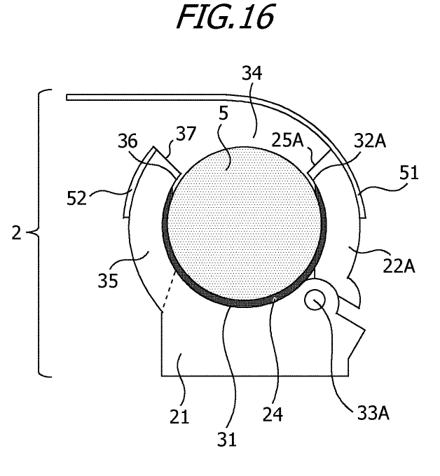

FIGS. 15A and 15B are diagrams each illustrating one example of the cuff 2 according to the third embodiment. In the cuff 2 depicted in FIGS. 15A, 15B, the movable units 22A, 22B are asymmetric in shape with respect to the axis of the finger 5 (bilateral asymmetry on the sheet surface of each of FIGS. 15A, 15B). The curved portion 32A of the movable unit 22A and the curved portion 32B of the movable unit 22B are asymmetric in shape with respect to the axis of the finger 5, and the length of the curved portion 32B of the movable unit 22B is larger than the length of the curved portion 32A of the movable unit 22A. The length of the curved portion 32B of the movable unit 22B is larger than the length of the curved portion 32A of the movable unit 22A, and hence, when the airbag 24 expands to compress the part of periphery of the finger 5, the cuff 2 is further restrained from coming off the finger 5. It is therefore possible to more surely fix the cuff 2 to the finger 5.

As depicted in FIG. 15A, the adjustment band member 51 is provided along the outer peripheral surface of the movable unit 22A, and one end of the adjustment band member 52 is provided along the outer peripheral surface of the movable unit 22B. The length of the adjustment band member 52 is larger than the length of the adjustment band member 51. The other end of the adjustment band member 52 is detachably fixed to the adjustment band member 51. An arbitrary position of the other end of the adjustment band member 52 is attachable to the adjustment band member 51. When attaching the cuff 2 to the finger 5, the other end of the adjustment band member 52 is detached from the adjustment band member 51, whereby the cuff 2 reaches the open state. The finger 5 is received inside of the cuff 2 in the open state of the cuff 2. The other end of the adjustment band member 52 is attached to the adjustment band member 51 while pulling the adjustment band members 51, 52, thereby decreasing the angles of the movable units 22A, 22B to the base unit 21. The finger 5 is received inside of the cuff 2, and the angles of the movable units 22A, 22B to the base unit 21 are decreased, whereby the cuff 2 is fixed to the finger 5. Accordingly, the cuff 2 is fixed to the finger 5 in such a state that the base unit 21 and the movable units 22A, 22B are attached to the periphery of the finger 5.

As depicted in FIG. 15B, one end of the adjustment band member 51 is provided along the outer peripheral surface of the movable unit 22A, and the adjustment band member 52 is provided along the outer peripheral surface of the movable unit 22B. The length of the adjustment band member 51 is larger than the length of the adjustment band member 52. The other end of the adjustment band member 51 is detachably fixed to the adjustment band member 52. An arbitrary position of the other end of the adjustment band member 51 is attachable to the adjustment band member 52. When attaching the cuff 2 to the finger 5, the other end of the adjustment band member 51 is detached from the adjustment band member 52, whereby the cuff 2 reaches the open state. The finger 5 is received inside of the cuff 2 in the open state of the cuff 2. The other end of the adjustment band member 51 is attached to the adjustment band member 52 while pulling the adjustment band members 51, 52, thereby decreasing the angles of the movable units 22A, 22B to the base unit 21. The finger 5 is received inside of the cuff 2, and the angles of the movable units 22A, 22B to the base unit 21 are decreased, whereby the cuff 2 is fixed to the finger 5. Consequently, the cuff 2 is fixed to the finger 5 in such a state that the base unit 21 and the movable units 22A, 22B are attached to the periphery of the finger 5.

The example of the configuration of the cuff 2 including one base unit 21, the plurality of movable units 22 (22A, 22B), the plurality of rotatable units 33 (33A, 33B) and the couple of adjustment band members 51, 52, has been described with reference to FIGS. 14 and 15. Described with reference to FIGS. 16 through 18 is an example of a configuration of the cuff 2 including one base unit 21, one movable unit 22 (22A), one rotatable unit 33 (33A) and the couple of adjustment band members 51, 52. FIG. 16 is a diagram illustrating one example of the cuff 2 according to the third embodiment. As illustrated in FIG. 16, the base unit 21 is provided with the protruded portion 35 taking the same shape as the shape of the movable unit 22B depicted in FIG. 14. In other words, the base unit 21 and the protruded portion 35 are integrally configured. The protruded portion 35 has the curved portion 36 extending along the part of periphery of the finger 5. The movable unit 22A and the protruded portion 35 are disposed so that the curved portion 32A of the movable unit 22A has the face-to-face relationship with the curved portion 36 of the protruded portion 35.

FIG. 16 depicts a state in which the cuff 2 is attached to the finger 5. The front end 25A of the movable unit 22A is not brought into contact with the front end 37 of the protruded portion 35, and the opening 34 is formed between the front end 25A of the movable unit 22A and the front end 37 of the protruded portion 35. Accordingly, the base unit 21, the protruded portion 35 and the movable unit 22A are attached to the finger 5 in the state of the opening 34 being formed along the part of periphery of the finger 5. In the cuff 2 illustrated in FIG. 16, the curved portion 32A of the movable unit 22A and the curved portion 36 of the protruded portion 35 are symmetric in shape with respect to the axis of the finger 5 (bilateral symmetry on the sheet surface of FIG. 16), and the length of the curved portion 32A of the movable unit 22A is the same as the length of the curved portion 36 of the protruded portion 35.

The airbag 24 is provided along at least the part of inner peripheral portion of the cuff 2, which is formed by the base unit 21, the movable unit 22A, the opening 34 and the protruded portion 35. The expansion of the airbag 24 compresses the part of periphery of the finger 5. The cuff 2 is fixed to the finger 5 by the adjustment band members 51, 52, and hence, when the airbag 24 expands to compress the part of periphery of the finger 5, the cuff 2 is restrained from coming off the finger 5.

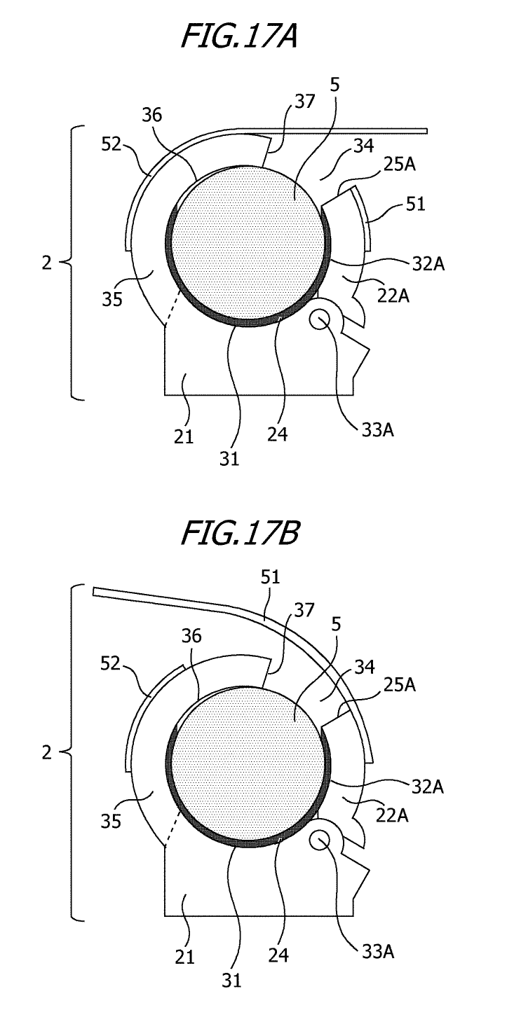

FIGS. 17A and 17B are diagrams each illustrating one example of the cuff 2 according to the third embodiment. In the cuff 2 depicted in FIGS. 17A, 17B, the curved portion 32A of the movable unit 22A and the curved portion 36 of the protruded portion 35 are asymmetric in shape with respect to the axis of the finger 5 (bilateral asymmetry on the sheet surface of each of FIGS. 17A and 17B), and the length of the curved portion 36 of the protruded portion 35 is larger than the length of the curved portion 32A of the movable unit 22A. The length of the curved portion 36 of the protruded portion 35 is larger than the length of the curved portion 32A of the movable unit 22A, and hence, when the airbag 24 expands to compress the part of periphery of the finger 5, the cuff 2 is further restrained from coming off the finger 5. It is therefore possible to more surely fix the cuff 2 to the finger 5.

As depicted in FIG. 17A, the adjustment band member 51 is provided along the outer peripheral surface of the movable unit 22A, and one end of the adjustment band member 52 is provided along the outer peripheral surface of the protruded portion 35. The length of the adjustment band member 52 is larger than the length of the adjustment band member 51. The other end of the adjustment band member 52 is detachably fixed to the adjustment band member 51. An arbitrary position of the other end of the adjustment band member 52 is attachable to the adjustment band member 51. When attaching the cuff 2 to the finger 5, the other end of the adjustment band member 52 is detached from the adjustment band member 51, whereby the cuff 2 reaches the open state. The finger 5 is received inside of the cuff 2 in the open state of the cuff 2. The other end of the adjustment band member 52 is attached to the adjustment band member 51 while pulling the adjustment band member 51, thereby decreasing the angle of the movable unit 22A to the base unit 21. The finger 5 is received inside of the cuff 2, and the angle of the movable unit 22A to the base unit 21 is decreased, whereby the cuff 2 is fixed to the finger 5. Consequently, the cuff 2 is fixed to the finger 5 in such a state that the base unit 21 and the movable unit 22A and the protruded portion 35 are attached to the periphery of the finger 5.

As depicted in FIG. 17B, one end of the adjustment band member 51 is provided along the outer peripheral surface of the movable unit 22A, and the adjustment band member 52 is provided along the outer peripheral surface of the protruded portion 35. The length of the adjustment band member 51 is larger than the length of the adjustment band member 52. The other end of the adjustment band member 51 is detachably fixed to the adjustment band member 52. An arbitrary position of the other end of the adjustment band member 51 is attachable to the adjustment band member 52. When attaching the cuff 2 to the finger 5, the other end of the adjustment band member 51 is detached from the adjustment band member 52, whereby the cuff 2 reaches the open state. The finger 5 is received inside of the cuff 2 in the open state of the cuff 2. The other end of the adjustment band member 51 is attached to the adjustment band member 52 while pulling the adjustment band member 51, thereby decreasing the angle of the movable unit 22A to the base unit 21. The finger 5 is received inside of the cuff 2, and the angle of the movable unit 22A to the base unit 21 is decreased, whereby the cuff 2 is fixed to the finger 5. Accordingly, the cuff 2 is fixed to the finger 5 in such a state that the base unit 21, the movable unit 22A and the protruded portion 35 are attached to the periphery of the finger 5.

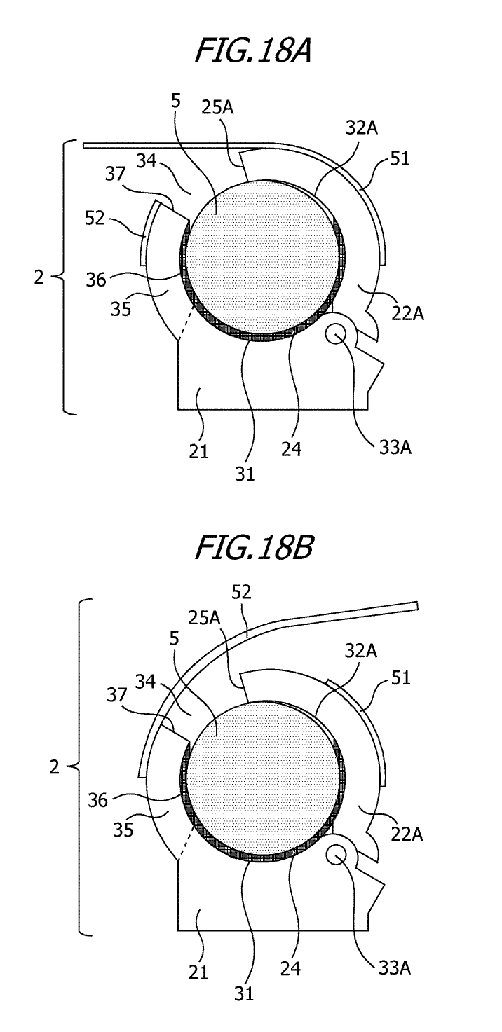

FIGS. 18A and 18B are diagrams each illustrating one example of the cuff 2 according to the third embodiment. In the cuff 2 depicted in FIGS. 18A, 18B, the curved portion 32A of the movable unit 22A and the curved portion 36 of the protruded portion 35 are asymmetric in shape with respect to the axis of the finger 5 (bilateral asymmetry on the sheet surface of each of FIGS. 18A and 18B), and the length of the curved portion 32A of the movable unit 22A is larger than the length of the curved portion 36 of the protruded portion 35. The length of the curved portion 32A of the movable unit 22A is larger than the length of the curved portion 36 of the protruded portion 35, and hence, when the airbag 24 expands to compress the part of periphery of the finger 5, the cuff 2 is further restrained from coming off the finger 5. It is therefore feasible to more surely fix the cuff 2 to the finger 5.

As depicted in FIG. 18A, one end of the adjustment band member 51 is provided along the outer peripheral surface of the movable unit 22A, and the adjustment band member 52 is provided along the outer peripheral surface of the protruded portion 35. The length of the adjustment band member 51 is larger than the length of the adjustment band member 52. The other end of the adjustment band member 51 is detachably fixed to the adjustment band member 52. An arbitrary position of the other end of the adjustment band member 51 is attachable to the adjustment band member 52. When attaching the cuff 2 to the finger 5, the other end of the adjustment band member 51 is detached from the adjustment band member 52, whereby the cuff 2 reaches the open state. The finger 5 is received inside of the cuff 2 in the open state of the cuff 2. The other end of the adjustment band member 51 is attached to the adjustment band member 52 while pulling the adjustment band member 51, thereby decreasing the angle of the movable unit 22A to the base unit 21. The finger 5 is received inside of the cuff 2, and the angle of the movable unit 22A to the base unit 21 is decreased, whereby the cuff 2 is fixed to the finger 5. Accordingly, the cuff 2 is fixed to the finger 5 in such a state that the base unit 21, the movable unit 22A and the protruded portion 35 are attached to the periphery of the finger 5.

As depicted in FIG. 18B, the adjustment band member 51 is provided along the outer peripheral surface of the movable unit 22A, and one end of the adjustment band member 52 is provided along the outer peripheral surface of the protruded portion 35. The length of the adjustment band member 52 is larger than the length of the adjustment band member 51. The other end of the adjustment band member 52 is detachably fixed to the adjustment band member 51. An arbitrary position of the other end of the adjustment band member 52 is attachable to the adjustment band member 51. When attaching the cuff 2 to the finger 5, the other end of the adjustment band member 52 is detached from the adjustment band member 51, whereby the cuff 2 reaches the open state. The finger 5 is received inside of the cuff 2 in the open state of the cuff 2. The other end of the adjustment band member 52 is attached to the adjustment band member 51 while pulling the adjustment band member 51, thereby decreasing the angle of the movable unit 22A to the base unit 21. The finger 5 is received inside of the cuff 2, and the angle of the movable unit 22A to the base unit 21 is decreased, whereby the cuff 2 is fixed to the finger 5. Accordingly, the cuff 2 is fixed to the finger 5 in such a state that the base unit 21, the movable unit 22A and the protruded portion 35 are attached to the periphery of the finger 5.

### Effects of Embodiments

In the cuff 2 according to each of the embodiments, when the cuff 2 is attached to the finger 5, the opening 34 is formed along the part of periphery of the finger 5. Accordingly, the airbag 24 expands to compress the part of periphery of the finger 5 in the state of the cuff 2 being partially opened, whereby a vein of the finger 5 is partially compressed. The blood pressure of the artery of the finger 5 is measured in the state of the cuff 2 being partially opened, and it is therefore feasible to restrain the finger 5 from being congested with the blood when measuring the blood pressure of the artery of the finger 5. The consecutive measurements of the blood pressures of the artery of the finger 5 in such a state that the entire periphery of the finger 5 is covered with the band-shaped cuff, cause the blood congestion of the finger 5 due to the excessive compression of the vein of the finger 5, and the measurement results are affected in some cases. The cuff 2 according to each embodiment enables restraint of the blood congestion of the finger 5, and enables accuracy of the measurement result to be improved.

The cuff 2 according to the first embodiment enables the angles of the movable units 22A, 22B to the base unit 21 to be adjusted by using the adjustment screws 23A, 23B. The cuff 2 according to the second embodiment enables the angles of the movable units 22A, 22B to the base unit 21 to be adjusted by using the adjustment springs 41A, 41B. The cuff 2 according to the third embodiment enables the angles of the movable units 22A, 22B to the base unit 21 to be adjusted by using the adjustment band members 51, 52. Consequently, the cuff 2 according to each embodiment enables the adjustment of the degree of how much the cuff 2 is fastened corresponding to thickness of the finger 5, and therefore, even when the finger 5 of each individual differs in thickness, it is feasible to measure the blood pressure of the artery of the finger 5. The cuff 2 according to the second embodiment facilitates the adjustments of the angles of the movable units 22A, 22B to the base unit 21 as compared with the cuffs 2 according to the first and third embodiments because of the variations of the angles of the movable units 22A, 22B to the base unit 21 when the finger 5 is received inside of the cuff 2.

What is claimed is:

1. A sphygmomanometer cuff for finger, comprising:
a base unit including a first curved portion extending along a part of periphery of a finger defined as a measurement examinee's target region;
at least one movable unit connected via a rotatable unit to the base unit, including a second curved portion extending along another part of periphery of the finger, and being attachable to the finger in a state of an opening being formed along the periphery of the finger;
an adjustment unit configured to adjust an angle of the movable unit to the base unit; and
a compression unit provided along at least a part of an inner peripheral portion formed along the periphery of the finger by the base unit, the movable unit and the opening, and compressing the part of periphery of the finger.

2. The sphygmomanometer cuff for finger according to claim 1, wherein the adjustment unit includes an adjustment screw.

3. The sphygmomanometer cuff for finger according to claim 1, wherein the adjustment unit includes an adjustment elastic body.

4. The sphygmomanometer cuff for finger according to claim 1, wherein the adjustment unit includes an adjustment band member.

5. The sphygmomanometer cuff for finger according to claim 1, wherein the at least one movable unit comprises a plurality of movable units including a first movable unit and a second movable unit,
the second curved portion of the first movable unit and the second curved portion of the second movable unit are asymmetric in shape with respect to an axis of the finger.

6. The sphygmomanometer cuff for finger according to claim 1, wherein the base unit includes a third curved portion extending along a part of periphery of the finger, and
the second curved portion of the movable unit and the third curved portion of the base unit are asymmetric in shape with respect to the axis of the finger.
